(19) United States
(12) Patent Application Publication
(10) Pub. No.: US 2018/0368339 A1
(43) Pub. Date: Dec. 27, 2018

(54) SOLID STATE SOIL MOISTURE SENSOR

(71) Applicant: Reinierus Hendricus Maria van der Lee, Temecula, CA (US)

(72) Inventor: Reinierus Hendricus Maria van der Lee, Temecula, CA (US)

(21) Appl. No.: 15/827,620

(22) Filed: Nov. 30, 2017

Related U.S. Application Data
(60) Provisional application No. 62/497,717, filed on Nov. 30, 2016.

(51) Int. Cl. A01G 25/16; G01N 33/24; H05K 5/06

(52) U.S. Cl. CPC A01G 25/167; H05K 5/069; G01N 33/246

(57) ABSTRACT

A soil moisture sensor includes a porous shell with an internal cavity. The soil moisture sensor includes a casted solid medium contained at least partially within the porous shell, where the solid medium is composed of granular particles and a binding agent such that the solid medium is porous. Electrodes in contact with the casted solid medium are spaced apart from one another, and are sized such that they extend beyond the perimeter of the porous shell. A cover is provided for the porous shell and adapted to allow electrical connection to the plurality of electrodes.

### CROSS REFERENCE TO RELATED PATENTS

The present U.S. Utility patent application claims priority pursuant to 35 U.S.C. § 119(e) to U.S. Provisional Application No. 62/497,717, entitled “SOLID STATE ELECTRICAL SENSOR FOR SENSING AND CONTROLLING MOISTURE IN SOILS”, filed Nov. 30, 2016, which is hereby incorporated herein by reference in its entirety and made part of the present U.S. Utility Patent Application for all purposes.

### BACKGROUND OF THE INVENTION

This invention relates generally to moisture sensing networks and more particularly to wireless sensor networks and sensors for use in such networks.

Networked sensors for measuring soil moisture are widely used to assist water management in large scale irrigation systems. Moisture sensors of varying complexity are often included in largescale irrigation systems that incorporate weather monitoring sensors and other moisture related data to reduce water usage, while maintaining or increasing crop yield. For example, moisture sensors can be used to detect soil moisture at different soil depths and locations within a crop area; the soil moisture can then be read by a microcontroller and transmitted over a network to be combined with weather data, crop data and other moisture data to determine watering needs for crop propagation. Accurate and precise moisture sensing are integral to water management systems striving for water savings while maintaining high crop yield.

### SUMMARY

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter. In an example embodiment, a soil moisture sensor includes a porous shell with an internal cavity. The soil moisture sensor includes a casted solid medium contained at least partially within the porous shell, where the solid medium is composed of granular particles and a binding agent such that the solid medium is porous. Electrodes in contact with the casted solid medium are spaced apart from one another, and are sized such that they extend beyond the perimeter of the porous shell. A cover is provided for the porous shell and adapted to allow for electrical connection to the plurality of electrodes.

### BRIEF DESCRIPTION OF THE SEVERAL VIEWS OF THE DRAWING(S)

A further understanding of the nature and advantages of the present invention may be realized by reference to the remaining portions of the specification and the drawings wherein like reference labels are used throughout the several drawings to refer to similar components. In some instances, reference labels include a numerical portion followed by a Latin-letter suffix; reference to only the numerical portion of reference labels is intended to refer collectively to all reference labels that have that numerical portion but different Latin-letter suffices. The invention will be described and explained with additional specificity and detail through the use of the accompanying drawing, in which:

FIG. 1 is schematic block diagram of a wireless moisture sensing system according to various embodiments of the present invention;

FIG. 2A is a cross-section view of a moisture sensor in accordance with the present invention;

FIG. 2B is a top view of a moisture sensor in accordance with the present invention;

FIG. 3 shows observed comparison of resistance to soil moisture response for different sensor materials;

FIG. 4 shows observed accuracy of soil sensor resistance as a function of various reference resistor values;

FIG. 5 shows observed sensor AC resistance as a function of soil water tension FIG. 6A is an external view of a solid-state sensor in accordance with the present invention;

FIG. 6B is a longitudinal view of a solid-state sensor in accordance with the present invention;

FIG. 7 depicts a moisture sensing environment in accordance with the present invention;

FIG. 8 is an exterior view of a moisture sensor in accordance with the present invention;

FIG. 9 is a schematic block diagram of an embodiment of a moisture sensing system in accordance with the present invention;

FIG. 10 is a schematic block diagram of a moisture sensing system integrated with an irrigation control system in accordance with the present invention;

FIG. 11 is a schematic block diagram of an irrigation control system in accordance with the present invention;

FIG. 12 is a logic diagram for ETo engine execution in accordance with the present invention.

FIG. 13 is illustrating a capacitive moisture sensor for use in a moisture sensor system.

### DETAILED DESCRIPTION OF SOME EXAMPLE EMBODIMENTS

According to an embodiment of the present invention, a novel solid-state moisture sensor is included in a moisture sensing network. The moisture sensing network uses soil moisture data detected by the solid-state moisture sensor in collaboration with predictive analytics (such as predictive modelling, machine learning, and data mining) of weather data, crop data, landscape data and other moisture data to provide a comprehensive management scheme for an irrigation system.

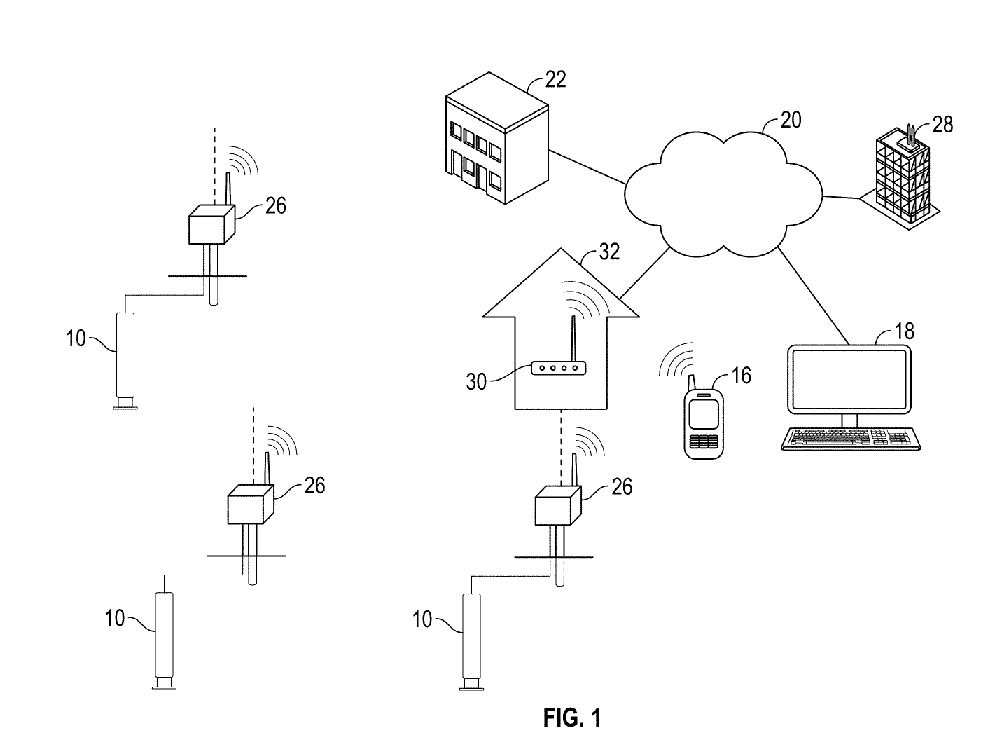

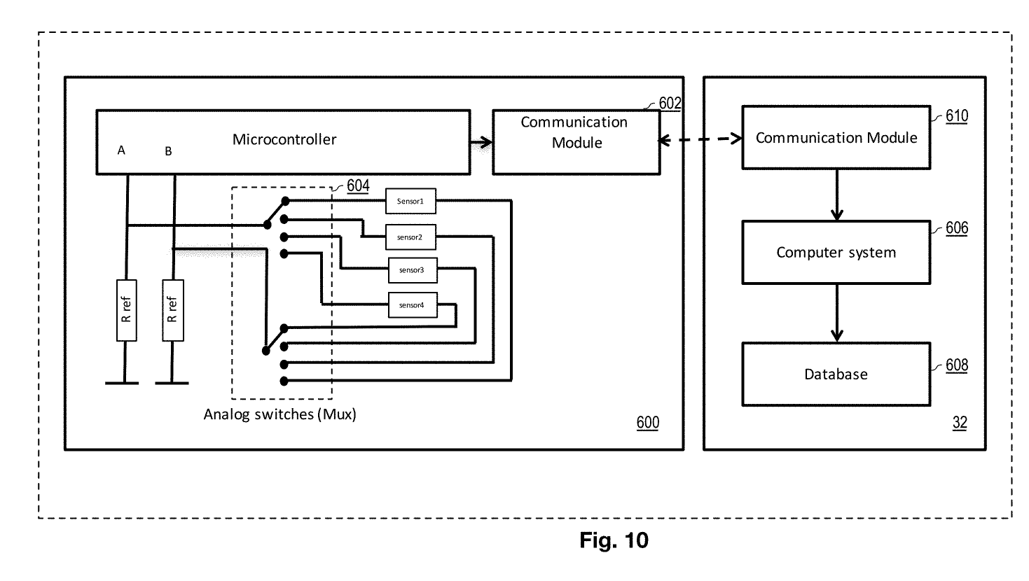

FIG. 1 is a schematic block diagram illustrating a wireless moisture sensing system according to various embodiments of the present invention. Gateway device 32 provides network access to a plurality of soil moisture sensors 10 located strategically over a crop landscape. Gateway device 32 is shown with wireless connection to transmitters 26, however connectivity can be accomplished over a wired network and/or hybrid wireless and wired network. Each soil moisture sensor 10 of FIG. 10 is shown connected to wireless transmitter 26. Alternatively, a single transmitter 26 could be connected to multiple soil moisture sensors 10, or transmitter 26 can be embedded in each soil moisture sensor 10. Wireless connectivity can be accomplished via a number of standard and proprietary networks. For example, long range, low power wireless platforms such as LoRa can provide for connectivity in the unlicensed ISM radio spectrum to a large number of sensors with battery and/or solar powered transceivers. Connectivity options include, but are not limited to other protocols intended for use in the ISM spectrum, such as WiLAN, Bluetooth, Zigbee and others. Additionally, with the potential for sensors to be widely distributed over a given landscape, sensors can be mesh-networked to increase range and provide robust networking options for harsh climates.

Implementation of LoRa communication can be using the lower communication layer, such as LoRa LAN, allowing proprietary private networks, or using standardized LoRaWAN networks that are being rolled out worldwide by network providers to enable low cost IoT device connectivity. Additionally, the wireless network provided by gateway 32 can be unsecured, such as can be the case in an unencrypted LoRaLAN network. Secure network options could include the use of the LoRaWAN protocol. Multiple options for connectivity will be evident to one skilled in the relevant art, including the use of a secure protocol over an otherwise unsecured network. For example, a network of LoRa radio modules and LoRa gateways can receive sensor station data from transmitter 26 and forward the data over cloud network 20 to and application servers at third party resource 28. The application of the LoRa standard wireless communication allows gateway and sensor stations to be miles apart, depending on local conditions and is therefore well suited for agricultural applications.

In an example embodiment, transmitter 26 is powered by an attached solar cell, and includes a microcontroller for reading the detected moisture from sensor 10 and controlling communication with gateway 32.

Gateway 32 can be connected to WAN network 20, providing connection to multiple third-party resources. For example, weather service 22 can be used to provide daily evapotranspiration data, along with short and long-term weather information for a modelling system running on a server 30 attached to gateway 22. Alternatively gateway 32 can provide moisture data collected from soil moisture sensors 10 to a third-party resource 28 for analysis and control of an irrigation system.

Modelling systems can used to correlate weather data with a specific crop information. For example, a modelling system can incorporate historical, current and predicted weather data, such as wind speed, temperature, solar radiation levels, barometric pressure, fog levels, etc., along with evapotranspiration data, specific crop data, soil data and soil moisture data to provide accurate and precise input to an irrigation system. Crop data can include canopy data, such as time-based measurements for canopy shade (measurement of percentage of light intercepted by a given plant's canopy) at different time in a growing season and at different times of the day. Crop spacing and crop type, along with a given crop's ideal soil tension range are also useful data for input to a modelling system. Additionally, a crop's ability to deal with deficit irrigation will also be a useful input for irrigation affected by less than ideal water availability.

Evapotranspiration, usually expressed as “reference evapotranspiration” (“ETo”), is a representation of the environmental demand for evapotranspiration and represents the evapotranspiration rate of a short green crop (usually grass), completely shading the ground, of uniform height and with adequate water status in the soil profile. ETo data is widely used to estimate the day water consumption use of a crop in order to irrigate the right amount to replenish that consumption.

The following equation can be used to calculate crop water requirements:

ETc=ETo×Kc

where ETc=crop evapotranspiration, ETo=reference evapotranspiration and Kc=crop coefficient. The above equation will give water requirements in inches (one acre inch=˜27,500 gallons per acre). The crop coefficient (Kc) is an indication of how much % of light is intercepted by the plant's canopy, it is dependent on the seasonal growth stage and the trellis design. You can determine the Kc by measuring the canopy shade width at noon.

By way of example, for an ETo of 0.25 inch, water consumption of a grape vine in a vineyard is:

ETc=0.25×0.375=0.0938

Daily use per acre=0.0938×27,500 gallons per acre=2,578 gallons

At 9×6 feet spacing: 806 vines/acre=3.2 gallons/vine/day
Irrigation time:
The vineyard has 2 drippers of 1 gallon/hr. per vine.
Required irrigation time for 3.2 gallon: 3.2/2×60=96 minutes

Returning to FIG. 1, soil moisture sensors 10 can be used with transmitter 26 and gateway 32 to calibrate the ETc system. For example, the ETo data can be compared with data collected from the soil moisture sensors to modify the ETo system and continually fine-tuned to provide improving accuracy.

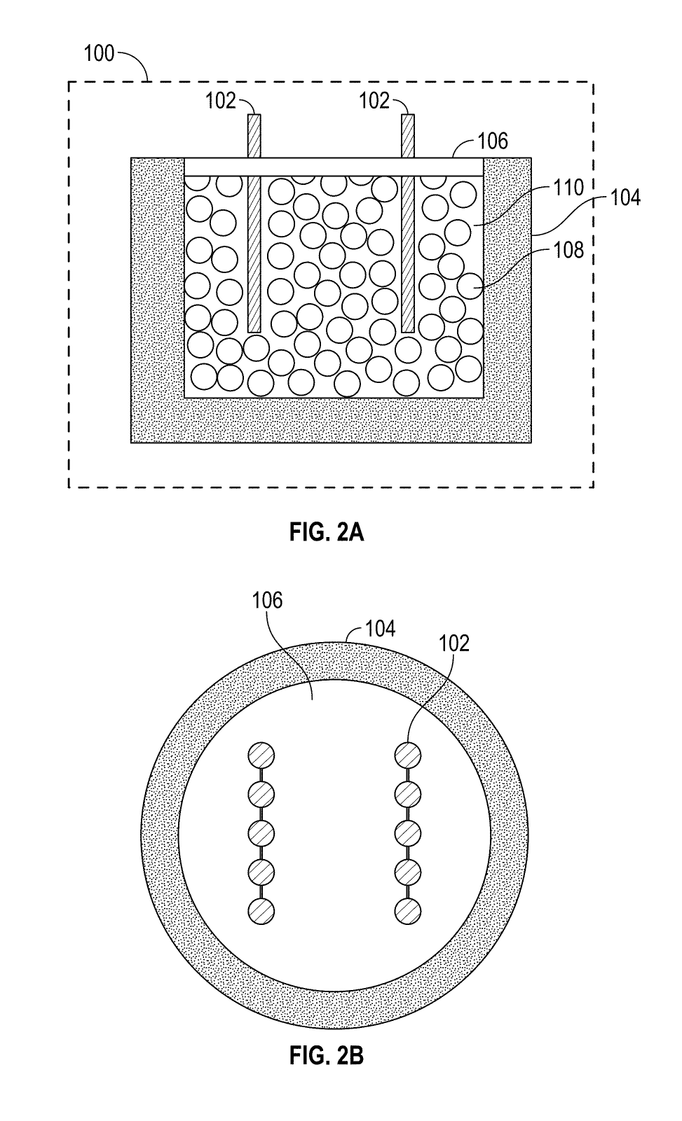

FIG. 2A is a cross-section view of the upper portion of an example soil moisture sensor. A porous shell 104 encases the bottom and cylindrical side of the sensor. Two electrodes 102 protrude through a cap 106 into the enclosed cavity created by shell 104, which comprises a casted solid-state medium 110. The encased solid-state medium 110 can be composed of gypsum or any other material to render the sensor less sensitive to soil salinity. The solid-state medium 110 can include binding agent 108 to provide mechanical strength and porosity around the electrodes 102. Solid-state medium 110 can be composed of any material that can provide free movement of moisture to the electrodes 102. For example, binding agent 108 can be one or more of micro-glass beads, Portland Cement, or volcanic glass in a paste of varying ratios with of one or more of gypsum, silica and perlite. The absorption and desorption (porosity) properties of solid media 108, together with properties of the binding agent 108 used to keep the particles together affects the resistance and/or capacitance value range for operational soil moisture conditions. FIG. 2B provides a top view of the upper portion of the example soil moisture sensor, with the electrodes 102 protruding through the cap 106 for connection to a microcontroller or other reading device.

Materials that can be mixed with the bonding agent to increase porosity and/or stability of the measurement cell medium include, but are not limited to Perlite, Diatomite, expanded clay, shale, pumice, slag and vermiculite. Based on the ratio of binding agent 108 to solid-state medium 110 volume a sensor can be designed to have an optimal measurement range for the soil moisture range of interest.

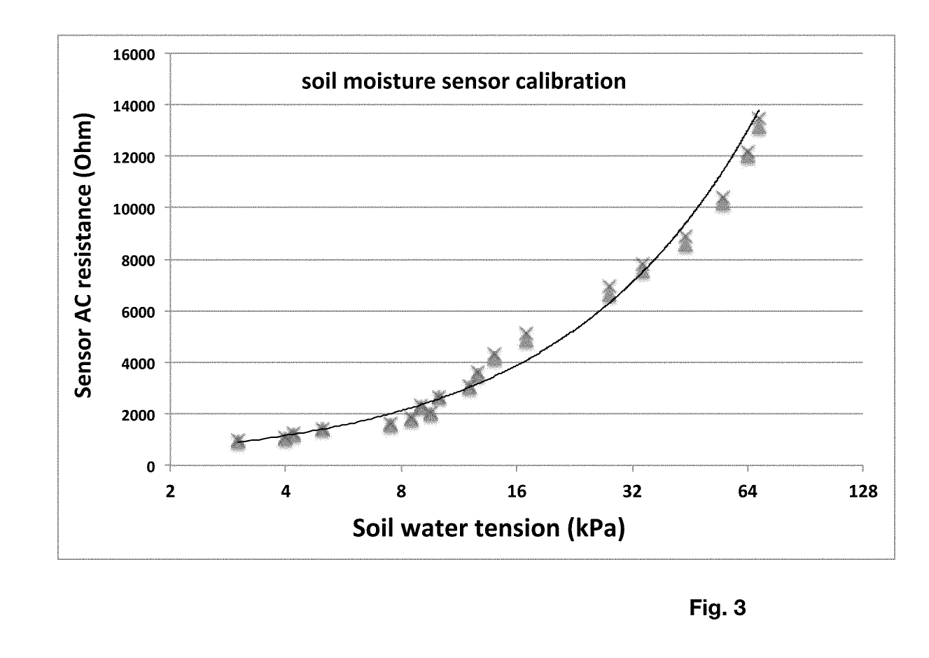

FIG. 3 shows observations of a sensor with a typical response to moisture levels of binding agent 108 to solid-state medium 110 sensors. The sensor exhibits an exponential relationship to resistance that is suitable for accurate calibration, where variations of the solid state medium can result in a different resistance range at similar soil moisture levels. Sensor resistance range can thus be optimized for the attached sensor electronics. For example, temperature related resistance influence in the electronics circuit (analog MUX switch series resistance) that can be largely eliminated by choosing a sensor with higher resistance output.

Electrodes can be composed of gold, or gold-plated bronze or other corrosion resistant material, such as stainless steel. Additionally, electrodes may be modified to provide enhanced surface area, such as, for example, porous materials, evacuated structures, or use designs such as multiple pins, concentric ring electrodes and metal mesh. Additional electrode materials can also be used, including carbon-based materials and other appropriate conductors.

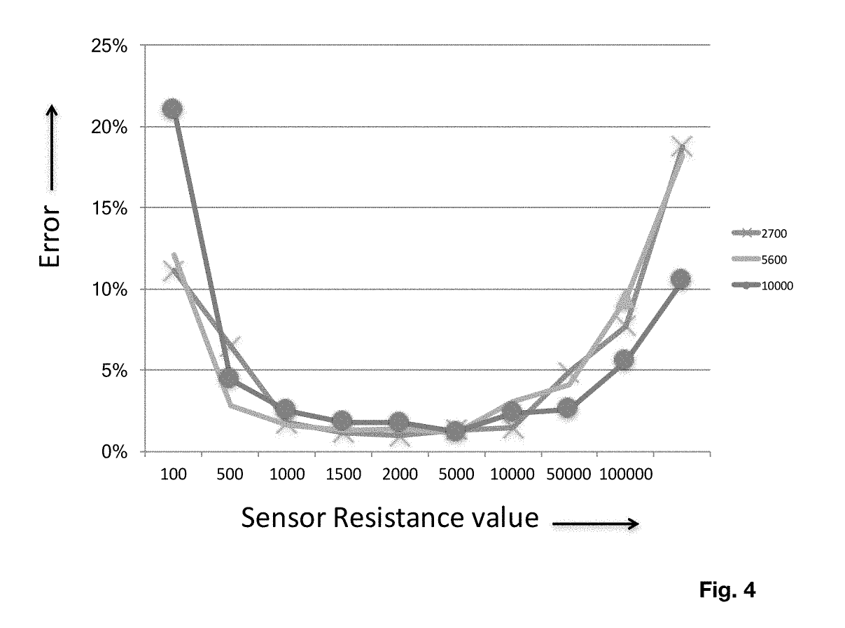

FIG. 4 shows observed accuracy of soil sensor resistance as a function of various reference resistor values. Resistance is determined by measuring the voltage on a voltage divider (sensor resistance and reference resistor), accordingly measuring at the middle of the supply voltage (where Rreference=Rsensor) provides the optimal ADC (analog to digital conversion) accuracy.

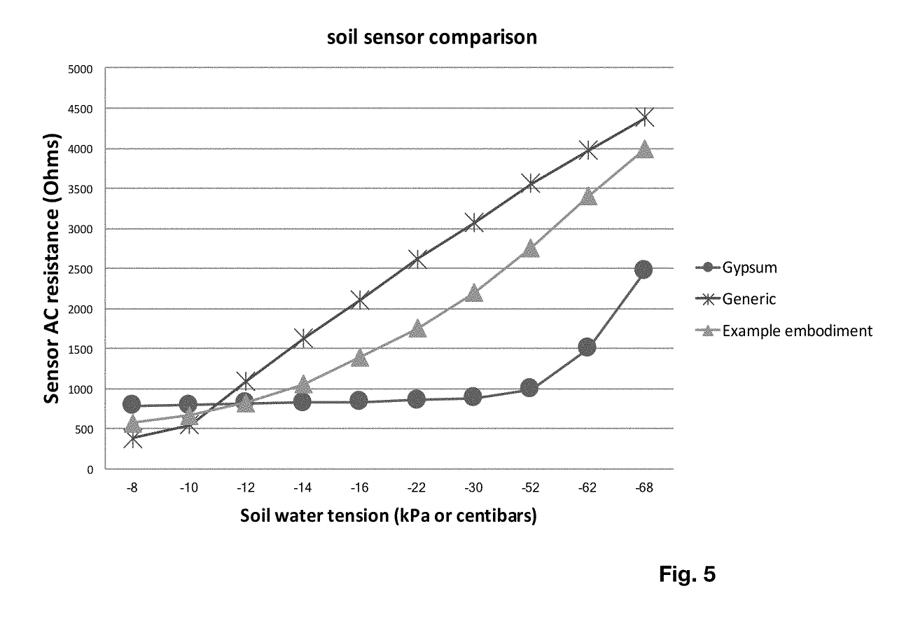

FIG. 5 shows observed sensor AC resistance as a function of soil water tension. Accuracy can be improved with temperature compensation. As observed, the sensor resistance of the gypsum block sensor only changes significantly at −55 kPa or higher. As most agricultural crops require an irrigation start point at higher moisture levels, gypsum sensors may be optimized for drought tolerant crops. In contrast, the generic sensor has a useful response starting at −10 kPa, accordingly, as the measurement graph shows, the example sensor provides a full range response from 0 to −75 kPa, making it suitable for all types of crops and soil types. Ratios and materials for solid-state medium 110 can be modified to accommodate the range of soil tension being measured (see FIGS. 3 and 5, below). In an example soil moisture sensor, a temperature sensor can be integrated; in another example soil temperature is sufficiently homogenous to place a temperature probe in the soil to achieve the same effect. In FIG. 3 a compensation of −3%/C provides acceptable goodness-of-fit (larger R²) to a trend curve.

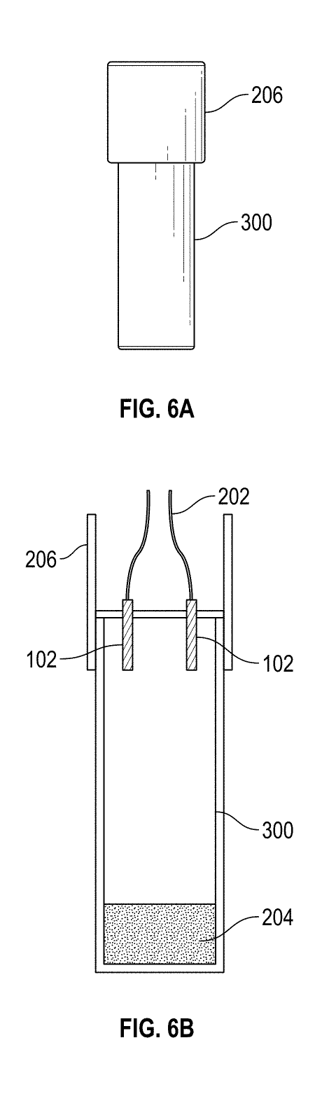

FIG. 6A provides an exterior view of an example moisture sensor, with a porous external shell 300 providing a cavity for the upper portion of the example soil moisture sensor, along with the bottom portion of the sensor. Porous external shell 300 can be composed of porous ceramic or any other material allowing free movement of moisture without dissolving over time in ground water. Porous external shell 300 allows solid state medium 110 from FIG. 2A around the electrodes to reach equilibrium with moisture in the surrounding soil, while providing mechanical strength and maintaining long term stable capillary contact with the surrounding soil. PVC tube 206 is adapted to firmly cover the top of porous external shell 300, providing structure for the intact moisture sensor, protection for electrical wiring, and helps with extraction of the sensor unit for service or replacement. For example, when tube 206 is PVC it can be expanded by controlled heating and then cooled to provide a firm connection to the sensor unit. Tube 206 can be comprised of any material suitable to the attributes attributed to PVC, including ABS, metal, fiberglass and other formable materials. Tube 206 can extend above the soil as necessary to place the wires out of access to rodents, other small animals and cultivation implements.

FIG. 6B provides a longitudinal cross section view of an example soil moisture sensor. Electrodes protrude into the upper portion of the moisture sensor, which is in-turn housed in porous external shell 300. Sensor portion 204 can be composed of structurally stabilized gypsum 204, or any other material allowing for capillary contact with surrounding soil. Structurally stabilized gypsum 204 (or equivalent) can act as a salinity buffer between the soil and the electrodes.

A given soil's “field capacity” is generally considered to be the amount soil moisture held in the soil after excess water has drained away. Field capacity will depend on the type of soil and/or the capillary forces on the soil. In practice water should move relatively freely through the porous external shell, saturating the structurally stabilized gypsum 204, along with the upper portion of the moisture sensor.

The drier a soil is the more vacuum force is required at a plant's roots to make use of the moisture in the soil. Soil moisture tension is a negative pressure (i.e. vacuum) measurement that indicates the pressure needed by the plant to extract water from the soil. Each crop will have an ideal range for soil moisture tension, which is dependent on the type of soil, the structure of the soil and the amount of salt contained in the soil. Accordingly, soil moisture sensors can be calibrated using a vacuum pump to simulate soil moisture tension, so that in a given time period the soil moisture sensor can be used to simulate the action of a crop's roots and plant stress due to water related effects.

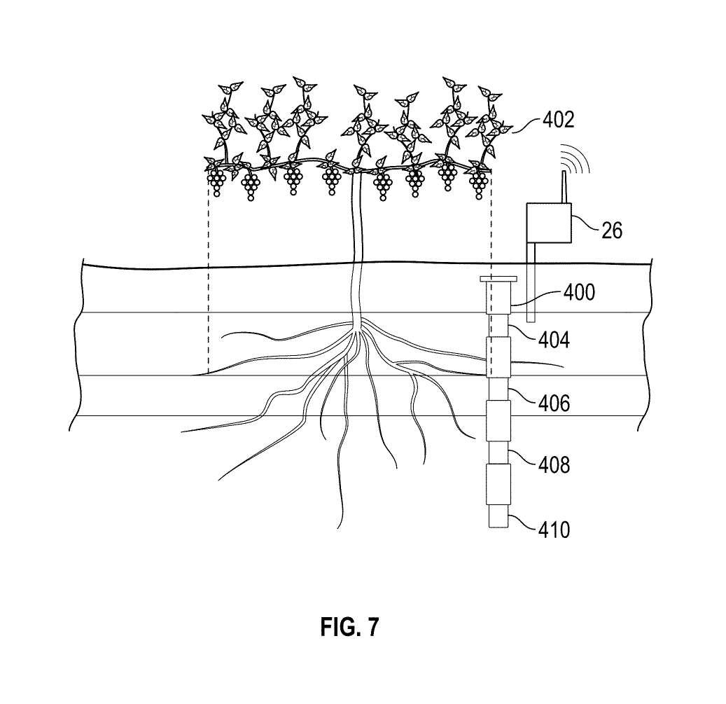

FIG. 7 illustrates a moisture sensing environment using an example four-part moisture sensor with transmitter 26. Four-part sensor array 400 includes sensors 404, 406, 408 and 410, where each porous section includes two electrodes and a channel to route wires out of sensor array 400. Each of sensors 404, 406, 408 and 410 are adapted to sense moisture at different soil levels. For example, section 404 can be placed at the 1 ft. level, with section 406 at the 2 ft. level and so on. In practice sensor 400 could include as few as two porous sections housing electrodes up to a practical limit, depending on depth and precision required. In an example, porous section 404 can be located just below the soil surface, while porous sections 406 and 408 can be located at a depth where roots are significantly involved in water uptake. Porous section 410 is then located at a depth below where roots are significantly involved in water uptake. Accordingly, moisture data can be provided to an irrigation system in order to improve water delivery efficiency.

Returning to FIG. 7, a bundle of electrode wires is attached to transmitter 26 as described in detail with respect to FIG. 1, above. Additionally, FIG. 7 illustrates a plant canopy perimeter with dashed lines extending to an example soil surface.

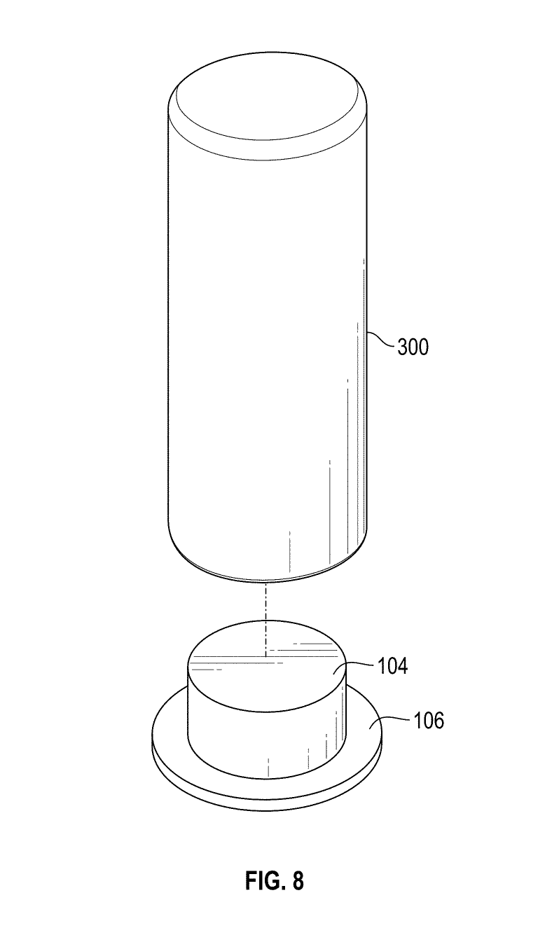

FIG. 8 is an external view of an example soil moisture sensor. Solid filler medium 104 encases the bottom and cylindrical sides of the sensor, which is adapted for insertion into porous external shell 300. Cap 106 is configured to act as a top cover for the sensor, providing a mechanical connection to porous external shell 300, as well as electrical connections for the sensor.

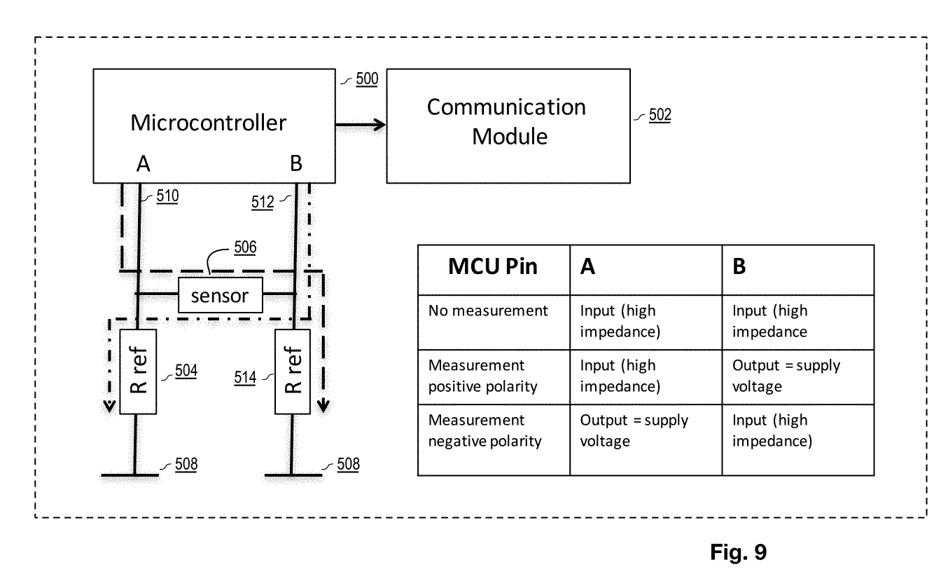

FIG. 9 provides a schematic block diagram of an example soil sensor interface to a microcontroller/transceiver. The example soil sensor interface uses alternating current direction to minimize electrolysis and gas forming effects on the sensor electrodes. Two microcontroller pins 510 and 512 are used for the sensor interface can be programmed as analog input (high impedance), and as output. When not in operation both sensor pins 508 are in input mode and no electric current flows through the sensor. For one single measurement, pin 510 is switched to output mode and provides a supply voltage to sensor 506. The sensor with reference resistors 504 and 514 form a voltage divider, and resistance at the sensor can be calculated from the voltage measured on analog input 512. After measurement, pin 510 is switched back to input mode. After the first measurement there is a timing delay, allowing for the electrolytes in the sensor to settle. For a second measurement (in the opposite direction), pin 512 is switched to output mode and provides a supply voltage to the sensor. The sensor with reference resistor 504 forms a voltage divider, and the sensor resistance can be calculated from the voltage measured on pin 510. After measurement, pin 512 is switched back to input mode. The two measurement results, made with opposite current direction, will be averaged by software to compensate for the concentration cell effect, where an undesired bias voltage is generated by the sensor cell.

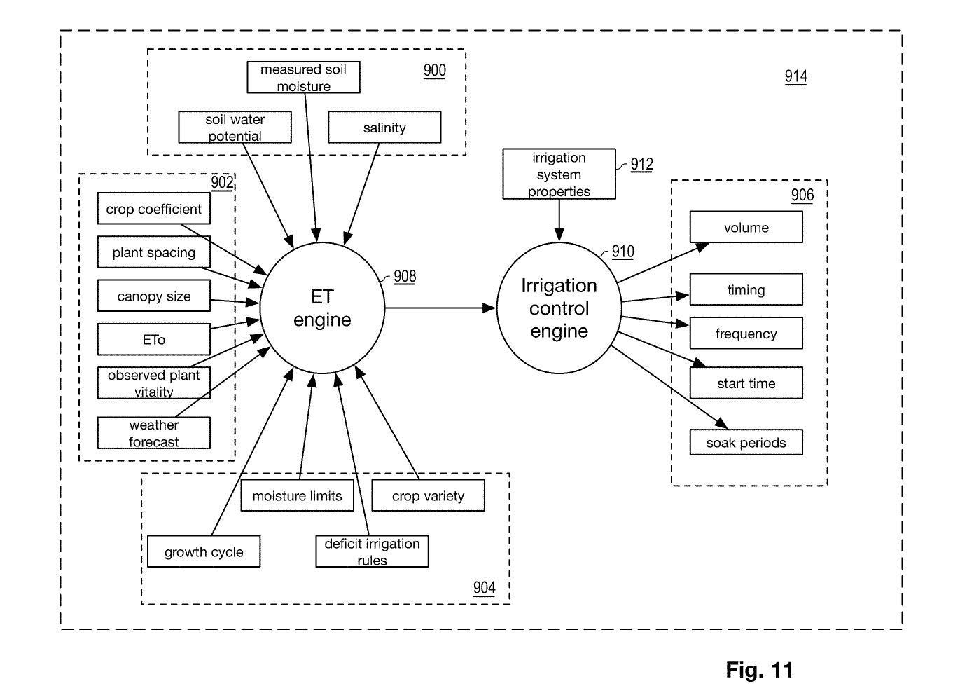

FIG. 10 is a schematic block diagram of an example soil sensor interface modified to support multiple soil moisture sensors and connected to a gateway. Sensor station 600 transmits data to gateway 32 from FIG. 1, which can forward sensor data directly to an Internet based service, also as illustrated in FIG. 1, or store the data locally in database storage 608 and process data locally using computer system 606. In an example, gateway 32 can provide control for irrigation valves (not shown), with control signals coming from an Internet based service (such as service 28 from FIG. 1), or by executing control locally using computer system 606. Sensor station 600 can include a multi-sensor array, as depicted in FIG. 7, wherein an analog mux 604 is used to collect sensor measurements from the sensor array, and electrically isolate sensors from being coupled due to soil resistance. Collected sensor data is transmitted via communication module 602 to communication module 610 associated with gateway 32. Irrigation control can be based on generic distributed control process(es) and/or use expert or machine learning systems. In an example of a machine learning system, input from the moisture sensor network and other resources is used to provide feedback to a distributed irrigation control process shown with further detail in FIG. 11.

FIG. 11 depicts a schematic of a comprehensive feedback compensated irrigation control system 914 with multiple resource inputs. In an example, soil related measurements 900, are input to ET engine 908, along with ET model inputs 902 and plant dependencies (i.e. factors related to a particular crop) 904. Soil related measurements 900 include at least one of measured soil moisture, salinity and soil water potential, which is a measure of the equivalent energy required make water available to the plant. Said another way it is a measure of the suction force required to extract water from a given soil.

ET model inputs 902 include at least one of crop coefficient (a calculated variable based on plant specific factors), plant spacing, canopy size (the area under plant canopy), ETo (reference evapotranspiration), observed plant variety (based on actual observation and/or machine analysis based image sensor input) and various forms of weather information, such as temperature, barometric pressure, wind, humidity, etc.). Plant dependencies 904 are the variables related to a specific crop. For example, a tree grower (such as an avocado farmer) will need much higher soil moisture for plant vitality than a wine grape grower. In another example, dependent on variety, in drought conditions, plants can be watered at deficit levels for periods of time without significant injury.

ET engine 908 provides input to irrigation control engine 910, which also receives input from irrigation system properties 912 and based on the inputs, calculates irrigation control engine output 906. Irrigation system properties 912 include at least one of delivery methods, delivery efficiency, irrigation capacity and distribution efficiency (measure of departure from homogenous delivery). Irrigation control engine output 906 can include, but is not limited to timing, volume, start time, required soak periods and volume. Volume, for example, can be the product of irrigation method capacity (for example a dripper with 1 gallon/hour capacity) per plant for a given operating period. For example, the volume for 1 gph dripper operated for one hour is 1 gallon. The daily volume needed per plant is calculated by the ET engine (908). Timing refers to the period of time during which water will be flowing, whereas frequency is the date and/or period when watering commences. Irrigation volume can be calculated on a daily basis; accordingly, irrigation volume would be the sum of daily volumes calculated over the number of days between irrigation events. Start time is the time of day when watering commences and allows the irrigation system to, for example, to reduce evaporation losses and/or minimize mold and fungus effects from sitting water. Soak periods are used to interrupt irrigation for a determined time period to allow soil to absorb the irrigation water and/or allow sufficient oxygen in the soil in order not to drown aerobic bacterial life. Additionally, some soil compositions, such as clay allow for higher watering efficiency when pre-soaks are used.

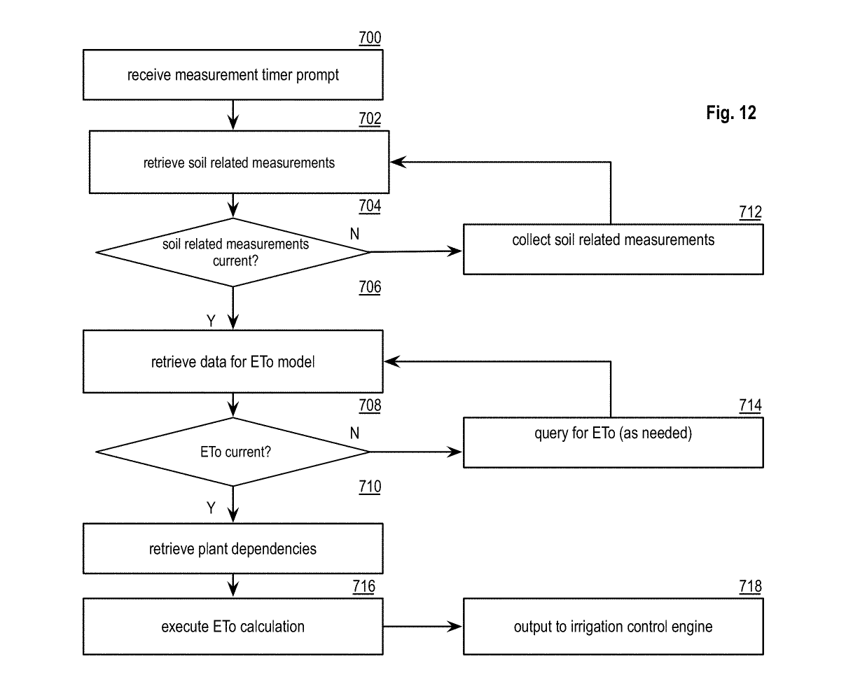

FIG. 12 is a logic diagram for ETo engine (or module) execution. The method beings at step 700, with the ETo module or engine being prompted to provide input to an irrigation control system. The prompting can be in the form of a timer, a query from the irrigation control system, a prompt from a user, or from a third party. The method continues at step 702, where the ETo engine retrieves soil related measurements detailed in FIG. 11. The retrieval can be one or more of a simple fetch from a memory storage (such as a buffer), a response to a query of soil measurement devices, and/or accessing a third-party application. At step 704 the ETo engine determines whether the soil related measurements are current, and in cases where the data can be stale, it may execute a collection routine at step 712 to update the soil related measurements and input them for retrieval by the ETo engine.

When soil related measurements are current, the method continues at step 706, where the ETo engine retrieves data for the ETo model, as detailed in FIG. 11. If ETo model data is not current, the ETo engine queries for updated ETo data in step 714. When the ETo data is current, the ETo engine continues by retrieving plant dependencies, as shown in step 710 and executes the ETo calculation in step 716. The ETo engine then provides input to the Irrigation control system (or engine) in step 718.

Importantly, steps 702, 706 and 710, along with associated subroutines may be executed in any order (not necessarily in the order of FIG. 12). Accordingly, inputs for ETo engine 908 from FIG. 11 can be received in any order desired without significant impact on the effectiveness of the overall system.

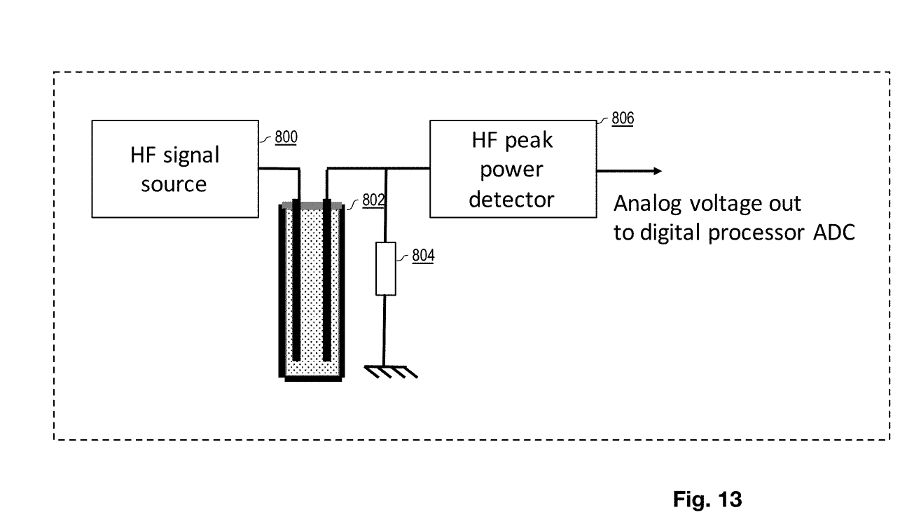

FIG. 13 provides a schematic diagram for an example capacitive moisture sensor in a moisture sensor system. Capacitive or frequency demain reflectometer (FDR) and time domain reflectometer (TDR) soil moisture sensors are based on soil dielectric properties that are dependent on the moisture content of a given soil. Since these sensors measure the volumetric water content and the water availability for plants based on this measurement is soil type specific, capacitive and TDR sensors require calibration to local soil composition. In contrast, resistive type sensors, such as those described above, measure the equivalent of soil water tension (how much suction force is needed to extract water from the soil), which is independent of soil composition. Sensor 802 uses a porous ceramic enclosure and a porous solid-state measurement medium to allow water to enter the sensor and come to equilibrium with the surrounding soil water content. A high frequency (HF) source 800 (excitation frequency, usually >30 MHz) is applied to a first electrode, which is the electrical equivalent of a capacitor plate. The second electrode (or capacitor plate) is connected to ground through resistor 804 and an HF peak power detector is used to measure the change in power dependent on the capacitance between the first and second electrode. Because the example sensor uses electrodes (capacitance plates) with an internal fringe field, this sensor can overcome typical disadvantages associated with capacitive sensors, thus sensitivity to local variations in the soil can be attenuated. Current state-of-art capacitive soil moisture sensors use external electrodes, where the volume of measured soil is determined by the size of the electric field. This is usually less than 5 cm range. The example sensor equalizes internal moisture with the surrounding soil moisture and therefore has the same large measurement volume as a resistive sensor.

In the example sensor, the charge Q on the capacitive sensor can be expressed using the following formula:

Q=CV, where C is the capacitance and V is the supply voltage.
Now since the current involved during the process is given as:

I=Q×F, where F is the frequency of the HF signal source.
Replacing Q=CV in the above formula, we get:

I=CVF, shows that the capacitance C is directly proportional to the current I, provided, the frequency and voltage is constant.

This magnitude of current will flow though the resistor and the voltage drop over the resistor can be rectified and buffered in a following HF peak power detector providing an analog output voltage that can be measured with an ADC for data processing.

In yet another example, the sensor illustrated in FIG. 2A can be combined with the capacitive sensor of FIG. 13 to measure both conductivity and capacitance of the soil being measured. Accordingly, since conductivity is necessarily dependent on the salinity and temperature of the water in the soil, and capacitance is the equivalent of the volumetric water content (and the volumetric water content is independent of salinity and temperature) the salinity of the soil can be “extracted” from the measurement. Salinity of the water under measurement can then be used as an independent data input for an irrigation control system, such as the system depicted in FIG. 10.

As may be used herein, the terms “substantially” and “approximately” provides an industry-accepted tolerance for its corresponding term and/or relativity between items. Such an industry-accepted tolerance ranges from less than one percent to fifty percent and corresponds to, but is not limited to, component values, integrated circuit process variations, temperature variations, rise and fall times, and/or thermal noise. Such relativity between items ranges from a difference of a few percent to magnitude differences. As may also be used herein, the term(s) “operably coupled to”, “coupled to”, and/or “coupling” includes direct coupling between items and/or indirect coupling between items via an intervening item (e.g., an item includes, but is not limited to, a component, an element, a circuit, and/or a module) where, for indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”. As may even further be used herein, the term “operable to” or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item. As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

As may also be used herein, the terms “processing module”, “processing circuit”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, and/or processing unit may be, or further include, memory and/or an integrated memory element, which may be a single memory device, a plurality of memory devices, and/or embedded circuitry of another processing module, module, processing circuit, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, and/or processing unit executes, hard coded and/or operational instructions corresponding to at least some of the steps and/or functions illustrated in one or more of the Figures. Such a memory device or memory element can be included in an article of manufacture.

The present invention has been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claimed invention. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality. To the extent used, the flow diagram block boundaries and sequence could have been defined otherwise and still perform the certain significant functionality. Such alternate definitions of both functional building blocks and flow diagram blocks and sequences are thus within the scope and spirit of the claimed invention. One of average skill in the art will also recognize that the functional building blocks, and other illustrative blocks, modules and components herein, can be implemented as illustrated or by discrete components, application specific integrated circuits, processors executing appropriate software and the like or any combination thereof.

The present invention may have also been described, at least in part, in terms of one or more embodiments. An embodiment of the present invention is used herein to illustrate the present invention, an aspect thereof, a feature thereof, a concept thereof, and/or an example thereof. A physical embodiment of an apparatus, an article of manufacture, a machine, and/or of a process that embodies the present invention may include one or more of the aspects, features, concepts, examples, etc., described with reference to one or more of the embodiments discussed herein. Further, from figure to figure, the embodiments may incorporate the same or similarly named functions, steps, modules, etc., that may use the same or different reference numbers and, as such, the functions, steps, modules, etc., may be the same or similar functions, steps, modules, etc., or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of the various embodiments of the present invention. A module includes a processing module, a functional block, hardware, and/or software stored on memory for performing one or more functions as may be described herein. Note that, if the module is implemented via hardware, the hardware may operate independently and/or in conjunction software and/or firmware. As used herein, a module may contain one or more sub-modules, each of which may be one or more modules.

While particular combinations of various functions and features of the present invention have been expressly described herein, other combinations of these features and functions are likewise possible. The present invention is not limited by the particular examples disclosed herein and expressly incorporates these other combinations.

What is claimed is:

1. A sensor for sensing moisture in soil, comprising:
a porous shell having a wall forming an internal cavity;
a casted solid medium contained at least partially in the porous shell, wherein the casted solid medium comprises granular particles and a binding agent, and further wherein the casted solid medium is porous;
a plurality of electrodes in contact with the casted solid medium, wherein the plurality of electrodes are spaced apart from each another, and wherein the electrodes are sized such that they extend beyond a perimeter of the porous shell; and
a cover for the porous shell, wherein the cover is adapted to allow electrical connection to the plurality of electrodes.

2. The sensor of claim 1, wherein the granular particles comprise at least one of Perlite, Diatomite, glass beads, expanded volcanic glass, expanded clay, shale, pumice, slag and Vermiculite.

3. The sensor of claim 1, wherein the porous shell is composed at least partially of ceramic material.

4. The sensor of claim 1, wherein the binding agent is composed at least partially of resin based material, cementaceous material and polystyrene.

5. The sensor of claim 1, wherein each electrode of the plurality of electrodes comprises corrosion resistant material.

6. The sensor of claim 5, wherein the corrosion resistant material comprises at least one of gold, gold-plated bronze, stainless steel.

7. The sensor of claim 1, further comprising an electrical circuit electrically connected to the plurality of electrodes, wherein the electrical circuit is adapted to measure a conductivity between two or more of the plurality of electrodes.

8. The sensor of claim 1, further comprising an electrical circuit electrically connected to the plurality of electrodes, wherein the electrical circuit is adapted to measure a capacitance between two or more of the plurality of electrodes.

9. The sensor of claim 1, further comprising an electrical circuit electrically connected to the plurality of electrodes, wherein the electrical circuit is further electrically connected to a microcontroller.

10. The sensor of claim 1, further comprising an electrical circuit electrically connected to the plurality of electrodes, wherein the electrical circuit is adapted to measure a conductivity between two or more of the plurality of electrodes and further wherein the electrical circuit is adapted to measure a capacitance between two or more of the plurality of electrodes.

11. The sensor of claim 1, further comprising a temperature sensor, wherein the temperature sensor is adapted to measure a temperature of the casted solid medium.

12. The sensor of claim 1, wherein a ratio of granular particles to binding agent is adapted to allow capillary motion of water through the casted solid medium.

13. The sensor of claim 12, wherein a capillary motion of water through the casted solid medium is equivalent to the capillary motion of water through soil.

14. A sensor array for sensing moisture in soil, comprising:
a plurality of sensors, each sensor of the plurality of sensors comprising:
a porous shell, the porous shell having a wall forming an internal cavity;
a casted solid medium contained at least partially in the porous shell,
wherein the casted solid medium comprises granular particles and a binding agent, and further wherein the casted solid medium is porous;
a plurality of electrodes in contact with the casted solid medium, wherein the plurality of electrodes are spaced apart from one another, and wherein the electrodes are sized to extend beyond a perimeter of the porous shell;
a cover for the porous shell, wherein the cover is adapted to allow electrical connection to the plurality of electrodes; and
a plurality of connecting members, each connecting member of the plurality of connecting members comprising:
a rigid outer surface forming an elongated cylinder, wherein the elongated cylinder is adapted to convey electrical conductors, and further wherein the elongated cylinder is adapted to rigidly connect to one or more sensors of the plurality of sensors.

15. The sensor array of claim 14, further comprises:
an electrical circuit electrically connected to at least two electrodes of the plurality of electrodes of each sensor of the plurality sensors, wherein the electrical circuit is adapted to measure a conductivity between at least two electrodes of the plurality of electrodes of each sensor of the plurality sensors.

16. The sensor array of claim 14, further comprises:
an electrical circuit electrically connected to at least two electrodes of the plurality of electrodes of each sensor of the plurality sensors, wherein the electrical circuit is adapted to measure a capacitance between at least two electrodes of the plurality of electrodes of each sensor of the plurality sensors.

17. The sensor array of claim 14, further comprises:
an electrical circuit electrically connected to at least two electrodes of the plurality of electrodes of each sensor of the plurality sensors, wherein the electrical circuit is adapted to measure a capacitance between at least two electrodes of the plurality of electrodes of each sensor of the plurality sensors and further wherein the electrical circuit is adapted to measure a resistance between at least two electrodes of the plurality of electrodes of each sensor of the plurality sensors.

18. The sensor array of claim 14, further comprises:
an electrical circuit electrically connected to at least two electrodes of the plurality of electrodes of each sensor of the plurality sensors, wherein the electrical circuit is adapted to measure a capacitance between at least two electrodes of the plurality of electrodes of each sensor of the plurality sensors.

19. The sensor array for sensing moisture in soil of claim 14, wherein the granular particles comprise at least one of Perlite, Diatomite, glass beads, expanded volcanic glass, expanded clay, shale, pumice, slag and Vermiculite.

20. The sensor array for sensing moisture in soil of claim 14, wherein the binding agent is composed at least partially of resin based material, cementaceous material and polystyrene.
